(19) United States
(12) Patent Application Publication
(10) Pub. No.: US 2026/0241126 A1
(43) Pub. Date: Aug. 20, 2026

(54) CONTROL OF A HUMIDITY AND A TEMPERATURE OF A RESPIRATORY AIR FLOW

(71) Applicant: Loewenstein Medical Technology S.A., Luxembourg (LU)

(72) Inventor: Juergen SARTOR, Karlsruhe (DE)

(21) Appl. No.: 19/539,627

(22) Filed: Feb. 13, 2026

(30) Foreign Application Priority Data
Feb. 18, 2025 (DE) 102025106103.3

(51) Int. Cl. A61M 16/00; A61M 16/16; F24F 11/63; F24F 11/80

(52) U.S. Cl. CPC A61M 16/026; A61M 16/161; F24F 11/63; F24F 11/80

(57) ABSTRACT

A method for controlling a humidity and a temperature of a respiratory air flow comprises: controlling the humidity/temperature in a plurality of successive time intervals in humidity/temperature control loops, wherein in each time interval of a humidity/temperature control loop a setpoint humidity/temperature value and an actual humidity/temperature value are received, a humidity/temperature deviation is determined by comparing the setpoint and actual humidity/temperature values and at least one humidity/temperature control parameter for controlling a power of a humidification device for humidifying the respiratory air flow is adjusted in order to reduce the humidity/temperature deviation.

### CROSS-REFERENCE TO RELATED APPLICATIONS

The present application claims priority under 35 U.S.C. § 119 of German Patent Application No. 10 2025 106 103.3, filed Feb. 18, 2025, the entire disclosure of which is expressly incorporated by reference herein.

### BACKGROUND OF THE INVENTION

### 1. Field of the Invention

The invention relates to a method for controlling a humidity and a temperature of a respiratory air flow by means of a control unit of a respiratory air humidification system. The invention furthermore relates to a control unit, to a computer program and to a computer-readable medium for carrying out such a method, and to a respiratory air humidification system having such a control unit.

### 2. Discussion of Background Information

Ventilated patients may sometimes suffer from excessively dry airways. In such cases, the respiratory air to be inhaled may be additionally humidified with the aid of a respiratory air humidifier. Drying of the mucous membranes can therefore be avoided or at least alleviated. In order additionally to be able to regulate the temperature of the respiratory air humidified in this way, a respiratory air line which connects a respiratory air outlet of the respiratory air humidifier to a suitable patient interface (for example a mask or a nasal cannula) may be heatable by means of an additional electrical heating device (for example a hose heater). Parallel control of the humidity and the temperature of respiratory air flow conveyed through the respiratory air line may have certain attendant difficulties, depending on the respective ambient conditions.

In view of the foregoing, it would be advantages to have available a method which enables reliable control of the humidity and the temperature of the respiratory air flow even in the event of relatively strongly varying ambient conditions. It further would be advantageous to have available a control unit, a computer program and a computer-readable medium for carrying out such a method, as well as a corresponding respiratory air humidification system.

### SUMMARY OF THE INVENTION

In a first aspect, the invention provides a method for controlling a humidity and a temperature of a respiratory air flow by means of a control unit of a respiratory air humidification system.

The respiratory air humidification system comprises, in addition to the control unit, a respiratory air humidifier having a respiratory air inlet, a respiratory air outlet, a liquid container and an electrically controllable humidification device, which is formed in order to convert an aqueous liquid from the liquid container into water vapour and/or water droplets. The respiratory air humidifier is formed in order to humidify respiratory air, which is supplied at the respiratory air inlet, by mixing with the water vapour and/or the water droplets and to provide it in the thereby humidified state as the respiratory air flow at the respiratory air outlet. The respiratory air humidification system furthermore comprises an electrically controllable thermal regulation device, which is formed in order additionally to regulate the temperature of the respiratory air flow, a humidity sensor for recording the humidity of the respiratory air flow and a temperature sensor for recording the temperature of the respiratory air flow.

The method comprises: controlling the humidity of the respiratory air flow in a plurality of successive time intervals in a humidity control loop, wherein in each time interval of the humidity control loop a setpoint humidity value and an actual humidity value, which indicates a humidity of the respiratory air flow as recorded by means of the humidity sensor, are received, a humidity deviation is determined by comparing the setpoint humidity value with the actual humidity value and at least one humidity control parameter for controlling a power, with which the humidification device converts the liquid into the water vapour and/or the water droplets, is adjusted in order to reduce the humidity deviation; controlling the temperature of the respiratory air flow in a plurality of successive time intervals in a temperature control loop (for example in parallel with the control of the humidity in the humidity control loop), wherein in each time interval of the temperature control loop a setpoint temperature value and an actual temperature value, which indicates a temperature of the respiratory air flow as recorded by the temperature sensor, are received, a temperature deviation is determined by comparing the setpoint temperature value with the actual temperature value and at least one temperature control parameter for controlling a power, with which the thermal regulation device additionally regulates the temperature of the respiratory air flow, is adjusted in order to reduce the temperature deviation.

The at least one humidity control parameter is adjusted (for example in at least one or each of the time intervals of the humidity control loop) while additionally taking into account the temperature deviation and/or the at least one temperature control parameter from at least one of the time intervals of the temperature control loop.

In addition or alternatively, the at least one temperature control parameter is adjusted (for example in at least one or each of the time intervals of the temperature control loop) while additionally taking into account the humidity deviation and/or the at least one humidity control parameter from at least one of the time intervals of the humidity control loop.

In other words, the humidity control loop and the temperature control loop may be coupled with one another in order deliberately to influence interactions between the two control loops, so that a high quality of control is achieved overall. This can have an advantageous effect on the reliability and/or accuracy during the (parallel) control of the humidity and the temperature of the respiratory air flow, particularly in the event of relatively strongly varying ambient conditions, for example the ambient temperature, the ambient humidity or the ambient pressure.

A further advantage is that this does not necessarily require further items of information, for example relating to a flow rate of the respiratory air flow, the respective ambient temperature or the respective ambient pressure.

The fact that two different types of sensors are employed can also obviate the need to take into account or compensate for measurement errors such as typically occur when employing a plurality of sensors of the same type. Overall, this can simplify the implementation of the control.

Such control also does not require complex mathematical models in relation to the physical properties of the respiratory air humidifier and/or of a respiratory air line attached to the latter (for example a heatable ventilation hose). Rather, such control works independently (or at least substantially independently) of such models so long as the power of the humidification and/or thermal regulation device is sufficient.

The method may for example be computer-implemented.

“Humidity” may for example be understood as an absolute air humidity and/or a relative air humidity in relation to the respective ambient temperature.

“Respiratory air” may be understood as a single respiratory gas or a mixture of a plurality of respiratory gases. A “respiratory gas” may for example be understood as carbon dioxide, oxygen, nitrogen, water vapor, anaesthetic gas or combinations thereof.

The humidity deviation may for example be determined by taking a difference between the setpoint humidity value and the actual humidity value. In a corresponding way, the temperature deviation may for example be determined by taking a difference between the setpoint temperature value and the actual temperature value.

An “actual value”, as in “actual humidity value” or “actual temperature value”, may be understood as a measurement value and/or estimated value (for example an average value) determined by means of the respective sensor.

A “control parameter”, as in “humidity control parameter” or “temperature control parameter”, may for example be understood as one of the following parameters: an electric current which flows between the electrical terminals of the humidification or thermal regulation device; an electric voltage which is applied to the electrical terminals of the humidification or thermal regulation device; a frequency at which the humidification or thermal regulation device is switched alternately on and off; a time ratio between on-phases, in which the humidification or thermal regulation device is switched on, and off-phases in which the humidification or thermal regulation device is switched off. For example, the frequency may be a clock frequency and/or the time ratio may be a duty cycle in the context of a pulse-width modulation.

For example, the power of the humidification device may be increased by corresponding adjustment of the at least one humidity control parameter when the actual humidity value indicates an insufficient humidity in relation to the setpoint humidity value, and vice versa. The adjustment of the at least one humidity control parameter (in one or both directions) may take place while additionally taking into account a current temperature of the respiratory air flow (for example the respective actual temperature value), a current temperature deviation and/or a current power of the thermal regulation device (for example a current value of the respective temperature control parameter), in order to achieve an optimal approximation of the two actual values to their respective setpoint value.

In addition or alternatively, the power of the thermal regulation device may for example be increased by corresponding adjustment of the at least one temperature control parameter when the actual temperature value indicates a temperature that is too low in relation to the setpoint temperature value, and vice versa. The adjustment of the at least one temperature control parameter (in one or both directions) may take place while additionally taking into account a current humidity of the respiratory air flow (for example the respective actual humidity value), a current humidity deviation and/or a current power of the humidification device (for example a current value of the respective humidity control parameter), in order to achieve an optimal approximation of the two actual values to their respective setpoint value.

In principle, the adjustment of the aforementioned control parameters may take place so that the humidity and the temperature of the respiratory air flow respectively approximate a desired operating point on a corresponding characteristic curve and/or in a corresponding set of characteristic curves, for example an h, x diagram.

For example, the control of the humidity and/or of the temperature may take place with the aid of a PID control. Depending on the application, other types of control are nevertheless also possible.

A second aspect of the invention relates to a control unit which is configured to carry out the method described above and below.

The control unit may comprise data processing elements. The data processing elements may be implemented as hardware and/or software and/or may comprise a processor. The processor may be configured to carry out the (computer-implemented) method. In addition to the processor, the control unit may comprise at least one of the following data processing elements: a memory, a bus system for data communication between the memory and the processor, a data communication interface for wireless and/or wired data communication with peripheral devices. Alternatively, the control unit may be implemented exclusively as hardware, for example in the form of an ASIC module or FPGA module.

It is pointed out that features of the method described above and below may also be features of the control unit (and vice versa).

A third aspect of the invention relates to a respiratory air humidification system. The respiratory air humidification system comprises a respiratory air humidifier having a respiratory air inlet, a respiratory air outlet, a liquid container and an electrically controllable humidification device, which is formed in order to convert an aqueous liquid from the liquid container into water vapour and/or water droplets. The respiratory air humidifier is formed in order to humidify respiratory air, which is supplied at the respiratory air inlet, by mixing with the water vapour and/or the water droplets and to provide it in the thereby humidified state as a respiratory air flow at the respiratory air outlet. The respiratory air humidification system furthermore comprises a respiratory air line for attachment to the respiratory air outlet, so that the respiratory air flow is conveyed through the respiratory air line, an electrically controllable thermal regulation device, which is formed in order additionally to regulate the temperature of the respiratory air flow, a humidity sensor for recording a humidity of the respiratory air flow, a temperature sensor for recording a temperature of the respiratory air flow and a control unit as described above and below.

The respiratory air inlet may for example be attachable to a ventilator for the invasive and/or non-invasive ventilation of a patient (or more precisely to a pressure outlet of the ventilator), in order to enable humidification of the respiratory air provided by the ventilator before the respiratory air is inhaled by the patient. A “ventilator” may also be understood as an anaesthesia device or a cough assist device for assisting a patient when coughing, also known as an insufflator-exsufflator.

A “respiratory air line” may for example be understood as a hose, a tube or a combination of these examples. The respiratory air line may be attachable with a first of its ends to the respiratory air outlet and/or with a second of its ends to a suitable patient interface-for example a mask, a nasal cannula and/or tubing. Accordingly, the first end may also be regarded as an end of the respiratory air line close to the respiratory air outlet and/or the second end may also be regarded as an end of the respiratory air line remote from the respiratory air outlet. In other words, the respiratory air outlet may be attachable to a patient's respiratory system via the respiratory air line and a suitable patient interface, so that the patient can be ventilated with the respiratory air flow.

“Water droplets” may be understood as particularly small water droplets of an aerosol. The water droplets may for example have an average particle size of about 50 μm or less, of about 10 μm or less, or of about 5 μm or less. On the other hand, the water vapour may be water in the gaseous state. The water vapour may be produced by deliberate (direct and/or indirect) heating and corresponding evaporation of the (aqueous) liquid.

For example, the humidification device and/or the thermal regulation device may comprise an electrical heating device. Such a heating device may for example be a heating resistor in the form of a heating rod, a heating filament, a heating plate or a combination of at least two of these examples.

In addition or alternatively, the humidification device may comprise an electrical atomization device for atomizing the liquid, i.e. for converting the liquid into (particularly small) water droplets, for example into an aerosol. Such an atomization device may for example be a piezoelectric ultrasonic atomizer.

The thermal regulation device may be formed in order additionally to regulate the temperature of the respiratory air flow directly and/or indirectly-for example by correspondingly regulating the temperature of at least one portion of the respiratory air line.

The thermal regulation device may also be formed in order to cool the respiratory air flow and/or at least one portion of the respiratory air line actively and/or passively. This has the advantage that both setpoint values may be achieved reliably even in the event of relatively high ambient temperatures and/or relatively large variations in the ambient temperature.

The control unit may be configured additionally to control one or more electropneumatic actuators for providing the respiratory air flow, for example in such a way that the humidity deviation is reduced and/or the temperature deviation is reduced. Such an electropneumatic actuator may for example be a fan or valve.

It is possible for the humidity sensor and the temperature sensor to be arranged in a common sensor housing. In other words, the humidity sensor and the temperature sensor may be embodied as different modules of one and the same sensor assembly. Such sensor assemblies are on the one hand particularly cost-effective and on the other hand particularly easy to install, for example on and/or in the respiratory air line.

The respiratory air humidifier may be embodied as an independent device and/or as an individual component (among a plurality of components) of a medical device, in particular of a ventilator.

Further aspects of the invention relate to a computer program and to a computer-readable medium, on which the computer program is stored.

The computer program comprises instructions which cause the control unit described above and below, for example a processor of the control unit, to carry out the method described above and below when the computer program is run by the control unit.

The computer-readable medium may be a volatile or nonvolatile data memory. For example, the computer-readable medium may be a hard disk drive, a USB storage device (universal serial bus), a RAM (random-access memory), a ROM (read-only memory), an EPROM (erasable programmable read-only memory), an EEPROM (electrically erasable programmable read-only memory), a flash memory or a combination of at least two of these examples. The computer-readable medium may also be a data communication network that makes it possible to download program code (for example via the Internet), or a cloud.

It should be pointed out that features of the method described above and below may also be features of the computer program and/or of the computer-readable medium (and vice versa).

Various embodiments of the invention are described below. These embodiments are not to be understood as a restriction of the scope of the invention.

According to one embodiment, the at least one humidity control parameter may be adjusted (for example in at least one or each of the time intervals of the humidity control loop) while additionally taking into account an earlier actual humidity value and/or an earlier humidity deviation from at least one earlier time interval of the humidity control loop which precedes the respective time interval. In other words, the at least one humidity control parameter may be adjusted (for example in at least one or each of the time intervals of the humidity control loop) while additionally taking into account a change (for example average and/or estimated) in the recorded humidity as a function of time and/or a change (for example average and/or estimated) in the humidity deviation as a function of time. This can further improve the accuracy during the control of the humidity, for example when determining the D-component of a PID control of the humidity. It is also conceivable for an average value to be determined from the (current) humidity deviation and from the earlier humidity deviation. The average value determined in this way may then, for example, be used in order to determine the I-component of a PID control of the humidity.

In addition or alternatively, the at least one temperature control parameter may be adjusted (for example in at least one or each of the time intervals of the temperature control loop) while additionally taking into account an earlier actual temperature value and/or an earlier temperature deviation from at least one earlier time interval of the temperature control loop which precedes the respective time interval. In other words, the at least one temperature control parameter may be adjusted (for example in at least one or each of the time intervals of the temperature control loop) while additionally taking into account a change (for example average and/or estimated) in the recorded temperature as a function of time and/or a change (for example average and/or estimated) in the temperature deviation as a function of time. This can further improve the accuracy during the control of the temperature, for example when determining the D-component of a PID control of the temperature. It is also conceivable for an average value to be determined from the (current) temperature deviation and from the earlier temperature deviation. The average value determined in this way may then, for example, be used in order to determine the I-component of a PID control of the temperature.

According to one embodiment, the at least one humidity control parameter may be adjusted (for example in at least one or each of the time intervals of the humidity control loop) while additionally taking into account the temperature deviation from at least one of the time intervals of the temperature control loop or while additionally taking into account both the temperature deviation and the at least one temperature control parameter from at least one of the time intervals of the temperature control loop, in order-in addition to the humidity deviation-to reduce the temperature deviation. In other words, the power of the humidification device (for example its heating power) may additionally be reduced correspondingly when the actual temperature value indicates a temperature that is too high in relation to the setpoint temperature value, and vice versa. In this way, an additional reduction or increase of the temperature of the respiratory air flow may be brought about.

In addition or alternatively, the at least one temperature control parameter (for example in at least one or each of the time intervals of the temperature control loop) while additionally taking into account the humidity deviation from at least one of the time intervals of the humidity control loop or while additionally taking into account both the humidity deviation and the at least one humidity control parameter from at least one of the time intervals of the humidity control loop, in order-in addition to the temperature deviation-to reduce the humidity deviation. In other words, the power of the thermal regulation device (for example its heating power) may additionally be reduced correspondingly when the actual humidity value indicates a humidity that is too low in relation to the setpoint humidity value, and vice versa. In this way, an additional reduction or increase of the humidity of the respiratory air flow may be brought about.

According to one embodiment, the at least one humidity control parameter may be adjusted in order additionally to reduce the temperature deviation only when it is discovered in the respective time interval of the humidity control loop that the thermal regulation device is deactivated and/or the temperature deviation reaches, for example exceeds or falls below, a determined threshold value. This can further improve the efficiency of the method.

In addition or alternatively, it is possible for the at least one temperature control parameter to be adjusted in order additionally to reduce the humidity deviation only when it is discovered in the respective time interval of the temperature control loop that the humidification device is deactivated and/or the humidity deviation reaches, for example exceeds or falls below, a determined threshold value. This can further improve the efficiency of the method.

A “threshold value” may be understood above and below as for example a positive or negative numerical value. In other words, a “threshold value” may be understood as a nonzero numerical value. By selecting a suitable threshold value, it is for example possible to prevent the respective control parameter from already being adjusted when the humidity deviation or temperature deviation is only very small. This can further improve the quality of control.

According to one embodiment, in each time interval of the humidity control loop and/or of the temperature control loop, a weighting value, which indicates a weighting of the humidity control loop in relation to the temperature control loop, may additionally be received. In this case, the adjustment of the at least one humidity control parameter and/or of the at least one temperature control parameter may take place in the respective time interval while additionally taking into account the weighting value. In this way, operating points that differ significantly from one another may even be set, for example in an h, x diagram, without excessively impairing the quality of control of the respectively lower-weighted control loop. For example, it is possible for the adjustment of the at least one humidity control parameter to take place only when the respective weighting value reaches a determined threshold value and/or the humidity control loop is allocated the same weighting or a higher weighting than the temperature control loop. The same may apply in a corresponding way for the adjustment of the at least one temperature control parameter.

According to one embodiment, the adjustment of the at least one humidity control parameter while additionally taking into account the weighting value may comprise: determining a first value range of possible values for the at least one humidity control parameter by using the weighting value and a first allocation rule, which allocates possible weighting values respectively to one of a plurality of possible first value ranges; adjusting the at least one humidity control parameter by using the first value range. In other words, possible settings for the at least one humidity control parameter may be deliberately limited or extended as a function of the respective weighting of the humidity control loop in relation to the temperature control loop.

A “value range”, as in “first value range” or “second value range” (see below), may be understood as a single positive or negative value (or the value zero) or a set of at least two different values.

An “allocation rule”, as in “first allocation rule” or “second allocation rule” (see below), may for example be understood as a mathematical function and/or a lookup table. Such an allocation rule may for example be stored in a memory of the control unit.

According to one embodiment, the adjustment of the at least one temperature control parameter while additionally taking into account the weighting value may comprise: determining a second value range of possible values for the at least one temperature control parameter by using the weighting value and a second allocation rule, which allocates possible weighting values respectively to one of a plurality of second value ranges; adjusting the at least one temperature control parameter by using the second value range. In other words, possible settings for the at least one temperature control parameter may be deliberately limited or extended as a function of the respective weighting of the temperature control loop in relation to the humidity control loop.

According to one embodiment, the setpoint humidity value may indicate a relative air humidity of about 100% or less, of about 98% or less, or of about 95% or less. With values that lie slightly below 100%, for example, sizeable inaccuracies during the control of the humidity may be avoided.

According to one embodiment, the setpoint temperature value may indicate a temperature of from about 30° C. to about 40° C., preferably from about 37° C. to about 39° C. At such temperatures, the inhaled respiratory air flow feels particularly comfortable. A respiratory air flow whose temperature is controlled in such a way can also particularly effectively alleviate or prevent drying of the mucous membranes.

According to one embodiment, the thermal regulation device may be formed in order additionally to regulate the temperature of the respiratory air flow by (for example active) warming and/or (for example active) cooling of at least one portion of the respiratory air line. This has the advantage that both setpoint values may be achieved reliably even in the event of relatively high ambient temperatures and/or relatively large variations in the ambient temperature.

According to one embodiment, the humidity sensor may be formed in order to record the humidity of the respiratory air flow at least at one humidity recording location inside an end portion of the respiratory air line remote from the respiratory air outlet. In addition or alternatively, the temperature sensor may be formed in order to record the temperature of the respiratory air flow at least at one temperature recording location inside an end portion of the respiratory air line remote from the respiratory air outlet. In both cases, the end portion may be a longitudinal portion of the respiratory air line, which extends (as seen in the longitudinal direction of the respiratory air line) starting from an end of the respiratory air line remote from the respiratory air outlet towards an end of the respiratory air line close to the respiratory air outlet, for example attached to the respiratory air outlet, over at most one third, at most one quarter, at most one tenth or at most one twentieth of a (total) length of the respiratory air line. This makes it possible to place the humidity sensor and/or the temperature sensor as close as possible to the respective patient. In this way, in comparison to an embodiment in which the humidity or temperature of the respiratory air flow is recorded in a different longitudinal portion of the respiratory air line outside the end portion, even in the event of relatively large variations in the ambient conditions, the actual humidity or temperature of the respiratory air flow shortly before it enters a patient interface attached to the respiratory air line can correspond with high accuracy to a desired humidity or temperature. In principle, it may be stated that the respective control error is commensurately less when the respective sensor is closer to the patient.

In addition or alternatively, the at least one humidity recording location and/or the at least one temperature recording location may lie in a cavity of a patient interface, for example a mask, a nasal cannula or tubing, attached to the respiratory air line, the respiratory air flow being conveyed into the cavity during operation of respiratory air humidification system from the respiratory air line so that the humidity and/or the temperature of the respiratory air flow can be recorded inside the cavity.

### BRIEF DESCRIPTION OF THE DRAWINGS

Embodiments of the invention are described below with reference to the appended drawings. Neither the description nor the drawings are to be understood as a restriction of the scope of the invention. In the drawings,

FIG. 1 shows a respiratory air humidification system according to one embodiment of the invention.

FIG. 2 shows a flowchart to illustrate a method according to one embodiment of the invention.

The drawings are purely schematic and not to scale. When the same references are used in various drawings, these references denote features that are identical or have the same effect.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

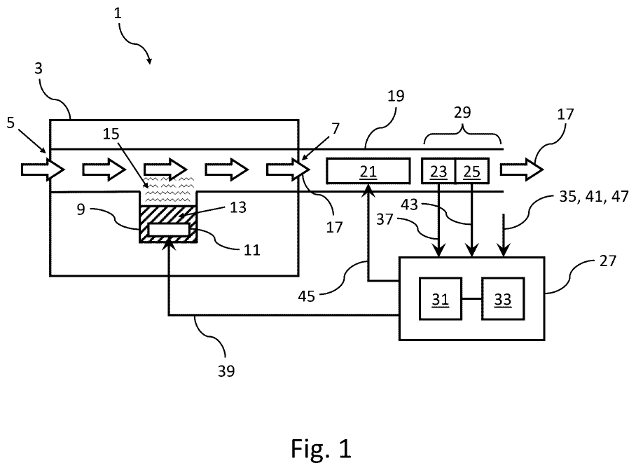

FIG. 1 shows a respiratory air humidification system 1. The respiratory air humidification system 1 comprises a respiratory air humidifier 3 having a respiratory air inlet 5, a respiratory air outlet 7, a liquid container 9 and an electrically controllable humidification device 11, here in the form of a first electrical heating device. In this example, the humidification device 11 is formed in order to convert an aqueous liquid 13 from the liquid container 9 into water vapour 15. The respiratory air humidifier 3 is formed in order deliberately to humidify respiratory air, which is supplied at the respiratory air inlet 5, by mixing with the water vapour 15 and to provide it in the thereby humidified state as a respiratory air flow 17 at the respiratory air outlet 7. Depending on the heating power of the humidification device 11, the respiratory air flow 17 which emerges at the respiratory air outlet 7 may be humid and/or warm to a varying degree.

The respiratory air humidification system 1 furthermore comprises a respiratory air line 19, which is attached with one of its ends to the respiratory air outlet 7 so that the respiratory air flow 17 is conveyed through the respiratory air line 19. The respiratory air line 19 may be attached with its other end, which is remote from the respiratory air outlet 7, to a patient interface, for example a mask, a nasal cannula or tubing, so that a patient can inhale the respiratory air flow 17 via the patient interface.

The respiratory air humidification system 1 also comprises an electrically controllable thermal regulation device 21, which is formed in order additionally to regulate the temperature of the respiratory air flow 17. The thermal regulation device 21 may be formed in order additionally to regulate the temperature of the respiratory air flow 17 by warming and/or cooling at least one portion of the respiratory air line 19. In this example, the thermal regulation device 21 is formed as a second electrical heating device for warming at least one portion of the respiratory air line 19.

The respiratory air humidification system 1 furthermore comprises a humidity sensor 23 for recording a humidity of the respiratory air flow 17, a temperature sensor 25 for recording a temperature of the respiratory air flow 17 and a control unit 27 for controlling the humidification device 11 and the thermal regulation device 21 by using sensor data of the two sensors 23, 25.

In this example, the humidity sensor 23 is formed in order to record the humidity of the respiratory air flow 17 at least at one humidity recording location inside an end portion 29 of the respiratory air line 19 remote from the respiratory air outlet 7. In a similar way, the temperature sensor 25 may be formed in order to record the temperature of the respiratory air flow 17 at least at one temperature recording location inside the same end portion 29. The end portion 29 may extend-as seen in the longitudinal direction of the respiratory air line 19-starting from the end of the respiratory air line 19 remote from the respiratory air outlet 7 towards an end of the respiratory air line 19 close to the respiratory air outlet 7 over at most one third, at most one quarter or at most one tenth of a length of the respiratory air line 19. This makes it possible to place the two sensors 23, 25 as close as possible to the patient.

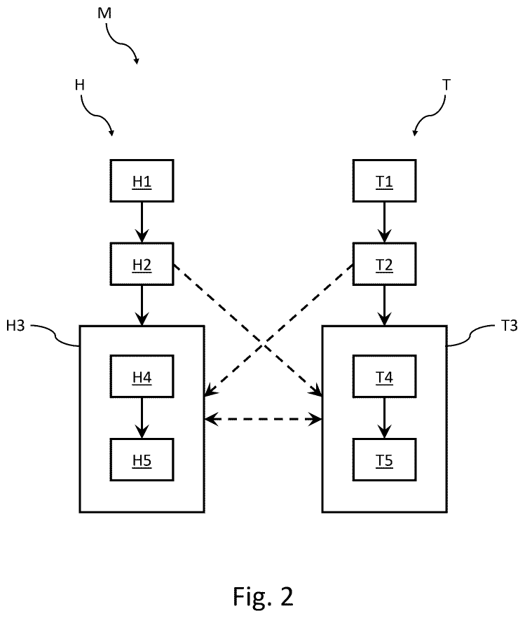

The control unit 27 is configured to carry out a method M (see FIG. 2) for controlling the humidity and the temperature of the respiratory air flow 17. For this purpose, the control unit 27 may for example comprise a processor 31 and a memory 33, in which a corresponding computer program may be stored. The processor 31 may be configured to run the computer program so that the method M is carried out.

The method M comprises on the one hand controlling the humidity of the respiratory air flow 17 in a plurality of successive time intervals in a humidity control loop H, and on the other hand controlling the temperature of the respiratory air flow 17 in a plurality of successive time intervals in a temperature control loop T, for example in parallel with the control of the humidity.

Each time interval of the humidity control loop h may comprise the following method steps.

In a step H1, a setpoint humidity value 35 and an actual humidity value 37, which indicates a humidity of the respiratory air flow 17 as recorded by means of the humidity sensor 23, are received. The setpoint humidity value 35 may for example indicate a relative air humidity of 100% or less, of 98% or less, or of 95% or less.

In a step H2, a humidity deviation is determined by comparing the setpoint humidity value 35 with the actual humidity value 37.

In a step H3, at least one humidity control parameter 39 for controlling a power, with which the humidification device 11 converts the liquid 13 into the water vapour 15, is adjusted in order to reduce the humidity deviation.

In a corresponding way, each time interval of the temperature control loop T may comprise the following method steps.

In a step T1, a setpoint temperature value 41 and an actual temperature value 43, which indicates a temperature of the respiratory air flow 17 as recorded by means of the temperature sensor 25, are received. The setpoint temperature value 41 may for example indicate a temperature of from about 30° C. to about 40° C.

In a step T2, a temperature deviation is determined by comparing the setpoint temperature value 41 with the actual temperature value 43.

In a step T3, at least one temperature control parameter 45 for controlling a power, with which the thermal regulation device 21 additionally regulates the temperature of the respiratory air flow 17, is adjusted in order to reduce the temperature deviation.

It is possible for the at least one humidity control parameter 39 to be adjusted in step H3 while additionally taking into account the temperature deviation and/or the at least one temperature control parameter 45 (or more precisely a setting of the respective temperature control parameter 45) from at least one of the time intervals of the temperature control loop T.

The respective humidity control parameter 39 may in this case be adjusted while additionally taking into account only the temperature deviation or while additionally taking into account both the temperature deviation and the respective temperature control parameter 45, in such a way that not only the humidity deviation but also (to a limited extent) the temperature deviation is reduced.

For example, the respective humidity control parameter 39 is adjusted in order thereby additionally to reduce the temperature deviation only when it is discovered in the respective time interval of the humidity control loop H that the thermal regulation device 21 is already deactivated and/or the temperature deviation has reached a determined threshold value.

In addition or alternatively, it is possible for the at least one temperature control parameter 45 to be adjusted while additionally taking into account the humidity deviation and/or the at least one humidity control parameter 39 (or more precisely a setting of the respective humidity control parameter 39) from at least one of the time intervals of the humidity control loop H.

The respective temperature control parameter 45 may in this case be adjusted while additionally taking into account only the humidity deviation or while additionally taking into account both the humidity deviation and the respective humidity control parameter 39, in such a way that not only the temperature deviation but also (to a limited extent) the humidity deviation is reduced.

For example, the respective temperature control parameter 45 is adjusted in order thereby additionally to reduce the humidity deviation only when it is discovered in the respective time interval of the temperature control loop T that the humidification device 11 is already deactivated and/or the humidity deviation has reached a determined threshold value.

It is also possible for a weighting value 47, which indicates a weighting of the humidity control loop H in relation to the temperature control loop T (for example a weighting of between 0% and 100%), additionally to be received in step H1 in each time interval of the humidity control loop H and/or in step T1 in each time interval of the temperature control loop T. In this case, the adjustment of the respective control parameter 39 or 45 in the respective time interval takes place while additionally taking into account the weighting value 47.

For example, the adjustment of the respective humidity control parameter 39 while additionally taking into account the weighting value 47 in step H3 may comprise a step H4 and a step H5. In step H4, a first value range of possible values for the respective humidity control parameter 39 may be determined by using the weighting value 47 and a first allocation rule, which allocates possible weighting values 47 respectively to one of a plurality of possible first value ranges. Next, in step H5, the adjustment of the respective humidity control parameter 39 may take place by using the first value range.

In a corresponding way, the adjustment of the respective temperature control parameter 45 while additionally taking into account the weighting value 47 in step T3 may comprise a step T4 and a step T5. In step T4, a second value range of possible values for the respective temperature control parameter 45 may be determined by using the weighting value 47 and a second allocation rule, which allocates possible weighting values 47 respectively to one of a plurality of possible second value ranges. Next, in step T5, the adjustment of the respective temperature control parameter 45 may take place by using the second value range.

The coupling of the two control loops H, T may for example involve the power of the humidification device 11, for example its heating power, being reduced in a suitable way by adjusting the at least one humidity control parameter 39 or the humidification device 11 being deactivated when the actual temperature value 43 exceeds the setpoint temperature value 41 and the power of the thermal regulation device 21, for example its heating power, is already zero (for example because the thermal regulation device 21 has already been deactivated).

Conversely, it is possible for the power of the thermal regulation device 21, for example its heating power, to be reduced in a suitable way by adjusting the at least one temperature control parameter 45 or for the thermal regulation device 21 to be deactivated when the actual humidity value 37 falls below the setpoint humidity value 35 (depending on the weighting of the two control loops H, T). The respiratory air line 19 may, for example, in this case also function as a passive cooling device. For this purpose, the respiratory air line 19 may additionally be formed with a corresponding heat sink, for example made of metal.

Finally, it should be pointed out that terms such as “have”, “comprise”, “include”, “with”, etc. do not exclude other elements or steps and that indefinite articles such as “a” or “an” do not exclude the plural.

Further, it should be pointed out that features or steps that are described with reference to one of the embodiments above may also be used in combination with features or steps that are described with reference to other embodiments among the embodiments above.

### LIST OF REFERENCE NUMERALS

1 respiratory air humidification system
3 respiratory air humidifier
5 respiratory air inlet
7 respiratory air outlet
9 liquid container
11 humidification device
13 liquid
15 water vapour
17 respiratory air flow
19 respiratory air line
21 thermal regulation device
23 humidity sensor
25 temperature sensor
27 control unit
29 end portion
31 processor
33 memory
35 setpoint humidity value
37 actual humidity value
39 humidity control parameter
41 setpoint temperature value
43 actual temperature value
45 temperature control parameter
47 weighting value
H humidity control loop
H1-5 steps of the humidity control loop
T temperature control loop
T1-5 steps of the temperature control loop

What is claimed is:

1. A method for controlling a humidity and a temperature of a respiratory air flow by a control unit of a respiratory air humidification system which comprises, in addition to the control unit:
a respiratory air humidifier having a respiratory air inlet, a respiratory air outlet, a liquid container and an electrically controllable humidification device, which is formed in order to convert an aqueous liquid from the liquid container into water vapour and/or water droplets, the respiratory air humidifier being formed in order to humidify respiratory air, which is supplied at the respiratory air inlet, by mixing with the water vapour and/or the water droplets and to provide it in a thereby humidified state as the respiratory air flow at the respiratory air outlet;
an electrically controllable thermal regulation device, which is formed in order additionally to regulate the temperature of the respiratory air flow;
a humidity sensor for recording the humidity of the respiratory air flow;
a temperature sensor for recording the temperature of the respiratory air flow;
wherein the method (M) comprises:
controlling the humidity of the respiratory air flow in a plurality of successive time intervals in a humidity control loop, wherein in each time interval of the humidity control loop a setpoint humidity value and an actual humidity value, which indicates a humidity of the respiratory air flow as recorded by the humidity sensor, are received, a humidity deviation is determined by comparing the setpoint humidity value with the actual humidity value and at least one humidity control parameter for controlling a power with which the humidification device converts the liquid into the water vapour and/or the water droplets, is adjusted in order to reduce the humidity deviation;
controlling the temperature of the respiratory air flow in a plurality of successive time intervals in a temperature control loop, wherein in each time interval of the temperature control loop a setpoint temperature value and an actual temperature value, which indicates a temperature of the respiratory air flow as recorded by the temperature sensor, are received, a temperature deviation is determined by comparing the setpoint temperature value with the actual temperature value and at least one temperature control parameter for controlling a power with which the thermal regulation device additionally regulates the temperature of the respiratory air flow, is adjusted in order to reduce the temperature deviation;
the at least one humidity control being adjusted while additionally taking into account the temperature deviation and/or the at least one temperature control parameter from at least one of the time intervals of the temperature control loop and/or
the at least one temperature control parameter being adjusted while additionally taking into account the humidity deviation and/or the at least one humidity control parameter from at least one of the time intervals of the humidity control loop.

2. The method of claim 1, wherein the at least one humidity control parameter is adjusted while additionally taking into account the temperature deviation from at least one of the time intervals of the temperature control loop or while additionally taking into account both the temperature deviation and the at least one temperature control parameter from at least one of the time intervals of the temperature control loop, in order additionally to reduce the temperature deviation, and/or
the at least one temperature control parameter is adjusted while additionally taking into account the humidity deviation from at least one of the time intervals of the humidity control loop while additionally taking into account both the humidity deviation and the at least one humidity control parameter from at least one of the time intervals of the humidity control loop, in order additionally to reduce the humidity deviation.

3. The method of claim 2, wherein the at least one humidity control parameter is adjusted in order additionally to reduce the temperature deviation only when it is discovered in the respective time interval of the humidity control loop that the thermal regulation device is deactivated and/or the temperature deviation reaches a predetermined threshold value, and/or
wherein the at least one temperature control parameter is adjusted in order additionally to reduce the humidity deviation only when it is discovered in the respective time interval of the temperature control loop that the humidification device is deactivated and/or the humidity deviation reaches a predetermined threshold value.

4. The method of claim 1, wherein in each time interval of the humidity control loop and/or of the temperature control loop a weighting value, which indicates a weighting of the humidity control loop in relation to the temperature control loop, is additionally received and an adjustment of the at least one humidity control parameter and/or of the at least one temperature control parameter takes place in the respective time interval while additionally taking into account the weighting value.

5. The method of claim 4, wherein the adjustment of the at least one humidity control parameter while additionally taking into account the weighting value comprises:
determining a first value range of possible values for the at least one humidity control parameter by using the weighting value and a first allocation rule, which allocates possible weighting values respectively to one of a plurality of possible first value ranges;
adjusting the at least one humidity control parameter by using the first value range.

6. The method of claim 4, wherein the adjustment of the at least one temperature control parameter while additionally taking into account the weighting value comprises:
determining a second value range of possible values for the at least one temperature control parameter by using the weighting value and a second allocation rule, which allocates possible weighting values respectively to one of a plurality of possible second value ranges;
adjusting the at least one temperature control parameter by using the second value range.

7. The method of claim 1, wherein the setpoint humidity value indicates a relative air humidity of 98%.

8. The method of claim 1, wherein the setpoint humidity value indicates a relative air humidity of 95% or less.

9. The method of claim 1, wherein the setpoint temperature value indicates a temperature of from 30° C. to 40 °C.

10. The method of claim 1, wherein the setpoint temperature value indicates a temperature of from 37° C. to 39° C.

11. A control unit for a respiratory air humidification system which comprises:
a respiratory air humidifier having a respiratory air inlet, a respiratory air outlet, a liquid container and an electrically controllable humidification device, which is formed in order to convert an aqueous liquid from the liquid container into water vapour and/or water droplets, the respiratory air humidifier being configured to humidify respiratory air, which is supplied at the respiratory air inlet, by mixing with the water vapour and/or the water droplets and to provide it in the thereby humidified state as a respiratory air flow at the respiratory air outlet;
an electrically controllable thermal regulation device, which is configured for additionally regulating the temperature of the respiratory air flow;
a humidity sensor for recording a humidity of the respiratory air flow;
a temperature sensor for recording a temperature of the respiratory air flow;
wherein the control unit configured for carrying out the method of claim 1.

12. A respiratory air humidification system, wherein the system comprises:
a respiratory air humidifier having a respiratory air inlet, a respiratory air outlet, a liquid container and an electrically controllable humidification device, which is configured to convert an aqueous liquid from the liquid container into water vapour and/or water droplets, the respiratory air humidifier being configured to humidify respiratory air, which is supplied at the respiratory air inlet, by mixing with the water vapour and/or the water droplets and to provide it in the thereby humidified state as a respiratory air flow at the respiratory air outlet;
a respiratory air line configured for attachment to the respiratory air outlet, so that the respiratory air flow is conveyed through the respiratory air line;
an electrically controllable thermal regulation device, which is configured to additionally regulate the temperature of the respiratory air flow;
a humidity sensor for recording a humidity of the respiratory air flow;
a temperature sensor for recording a temperature of the respiratory air flow;
the control unit of claim 11.

13. The respiratory air humidification system of claim 12, wherein the thermal regulation device is configured to additionally regulate the temperature of the respiratory air flow by warming and/or cooling at least one portion of the respiratory air line.

14. The respiratory air humidification system of claim 12, wherein the humidity sensor is configured to record the humidity of the respiratory air flow at least at one humidity recording location inside an end portion of the respiratory air line remote from the respiratory air outlet, and/or wherein the temperature sensor is configured to record the temperature of the respiratory air flow at least at one temperature recording location inside an end portion of the respiratory air line remote from the respiratory air outlet;
the end portion being a longitudinal portion of the respiratory air line, which extends starting from an end of the respiratory air line remote from the respiratory air outlet towards an end of the respiratory air line close to the respiratory air outlet over at most one third of a length of the respiratory air line.

15. The respiratory air humidification system of claim 14, wherein the end portion extends starting from an end of the respiratory air line remote from the respiratory air outlet towards an end of the respiratory air line close to the respiratory air outlet over at most one quarter of a length of the respiratory air line.

16. The respiratory air humidification system of claim 14, wherein the end portion extends starting from an end of the respiratory air line remote from the respiratory air outlet towards an end of the respiratory air line close to the respiratory air outlet over at most one tenth of a length of the respiratory air line.
